(19) United States
(12) Patent Application Publication
(10) Pub. No.: US 2024/0100050 A1
(43) Pub. Date: Mar. 28, 2024

(54) MDM2 INHIBITORS AND USES THEREOF

(71) Applicant: PMV Pharmaceuticals, Inc., Princeton, NJ (US)

(72) Inventors: Melissa DUMBLE, Watchung, NJ (US); Stephen JONES, Piscataway, NJ (US); Anna PUZIO-KUTER, Hillsborough, NJ (US); Binh VU, North Caldwell, NJ (US)

(21) Appl. No.: 18/351,108

(22) Filed: Jul. 12, 2023

Related U.S. Application Data
(60) Provisional application No. 63/388,479, filed on Jul. 12, 2022.

(51) Int. Cl. A61K 31/506; A61K 31/40; A61K 31/407; A61K 31/435; A61K 31/444; A61P 35/00

(52) U.S. Cl. CPC A61K 31/506; A61K 31/40; A61K 31/407; A61K 31/435; A61K 31/444; A61P 35/00

(57) ABSTRACT

Provided herein are methods and compositions of treating a condition by intratumorally administering MDM2 inhibitors. In some embodiments, the pharmaceutical compositions for intratumoral administration are formulated for controlled release and can comprise a biodegradable polymer.

### CROSS-REFERENCE TO RELATED APPLICATIONS

This application claims the benefit of U.S. Provisional Application No. 63/388,479, filed Jul. 12, 2022, which is incorporated herein by reference in its entirety.

### BACKGROUND

Cancer, an uncontrolled proliferation of cells, is a multifactorial disease characterized by tumor formation, growth, and in some instances, metastasis. Cells carrying an activated oncogene, damaged genome, or other cancer-promoting alterations can be prevented from replicating through an elaborate tumor suppression network. Murine double minute (MDM) family members MDM2 and MDMX can act as regulators of p53 and modulate cell cycle regulation and cell proliferation

### INCORPORATION BY REFERENCE

Each patent, publication, and non-patent literature cited in the application is hereby incorporated by reference in its entirety as if each was incorporated by reference individually.

### SUMMARY OF THE INVENTION

Disclosed herein is a method of treating a tumor in a subject in need thereof comprising administering to the subject a therapeutically-effective amount of a MDM2 inhibitor, wherein the administering is by intratumoral injection at most once every 3 days.

Disclosed herein is a method of treating a tumor in a subject in need thereof comprising administering to the subject a pharmaceutical composition, wherein the pharmaceutical composition comprises: a) a therapeutically-effective amount of a MDM2 inhibitor; and b) a polymer that degrades by hydrolysis in water, wherein the administering is by intratumoral injection.

Disclosed herein is a pharmaceutical composition comprising in a unit dosage form: a) a polymer that degrades by hydrolysis in water; and b) a therapeutically-effective amount of a MDM2 inhibitor, wherein the pharmaceutical composition is formulated for intratumoral injection.

### BRIEF DESCRIPTION OF THE FIGURES

FIG. 1 shows images of NUGC3 tumors injected intratumorally with blue dye. The dotted lines indicate the tumor mass that turned blue from the dye.

FIG. 2 shows the effects of intratumoral dosing regimens of navtemadlin in SJSA1 tumors, such as on tumor volume (mm³) and % body weight change.

### DETAILED DESCRIPTION

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although different cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

Tumor suppressor p53 is frequently inactivated by mutations or deletions in cancer. Murine double minute (MDM) family members MDM2 and MDMX (also known as HDM2 and HDMX) act as regulators of p53, a key controller of cell cycle regulation and cell proliferation. Alterations to MDM2 or MDMX can affect cancer development and progression. The interaction between tumor suppressor p53 and the E3 ligase MDM2 can be targeted to treat cancers with wild-type or functional TP53. Selective small-molecule MDM2 inhibitors have been identified, but their efficacy remains limited. New modes of administration, such as intratumoral administration, may significantly enhance the efficacy of MDM2 inhibitors and lead to better treatment of cancers.

Disclosed herein is a method of treating a tumor in a subject in need thereof comprising administering to the subject a therapeutically-effective amount of a MDM2 inhibitor, wherein the administering is by intratumoral injection at most once every 3 days.

Disclosed herein is a method of treating a tumor in a subject in need thereof comprising administering to the subject a pharmaceutical composition, wherein the pharmaceutical composition comprises: a) a therapeutically-effective amount of a MDM2 inhibitor; and b) a polymer that degrades by hydrolysis in water, wherein the administering is by intratumoral injection.

Disclosed herein is a pharmaceutical composition comprising in a unit dosage form: a) a polymer that degrades by hydrolysis in water; and b) a therapeutically-effective amount of a MDM2 inhibitor, wherein the pharmaceutical composition is formulated for intratumoral injection.

### MDM2 Inhibitor Compounds

Disclosed herein are pharmaceutical compositions comprising a MDM2 inhibitor. Also disclosed herein are methods of treating a condition in a subject in need thereof comprising administering a MDM2 inhibitor.

In some embodiments, the MDM2 inhibitor is as disclosed in US Publication No. 2008/0015194, PCT publication WO 2011/085126; PCT publication WO 2011/085129; PCT publication WO2011/076786, PCT publication WO2013/111105, PCT publication WO2011/098398, PCT publication WO/2018/178691, PCT publication WO/2018/161871, PCT publication WO/2017/060431, PCT publication WO/2017/055860, PCT publication WO/2017/055859, PCT publication WO/2016/028391, PCT publication WO/2016/026937, PCT publication WO/2016/001376, PCT publication WO/2016/028391, PCT publication WO/2016/026937, PCT publication WO/2016/001376, PCT publication WO/2014/151863, PCT publication WO/2014/134201, PCT publication WO/2014/130470, PCT publication WO/2014/115080, WO/2014/082889, PCT publication WO/2013/049250, PCT publication WO/2012/076513, PCT publication WO/2012/065022, PCT publication WO/2012/038307, PCT publication WO/2012/034954, PCT publication WO/2012/022707, and PCT publication WO/2011/153509.

In some embodiments, the MDM2 inhibitor is Nutlin-1, Nutlin-2, Nutlin-3, RG7112 (RO5045337), idasanutlin (RG7388), navtemadlin (AMG-232), APG-115, BI-907828, CGM097, siremadlin (HDM201), SAR405838 (MI-77301), MK-8242 (SCH 900242), milademetan (DS-3032b), or sulanemadlin (ALRN-6924), or a pharmaceutically-acceptable salt of any of the foregoing:

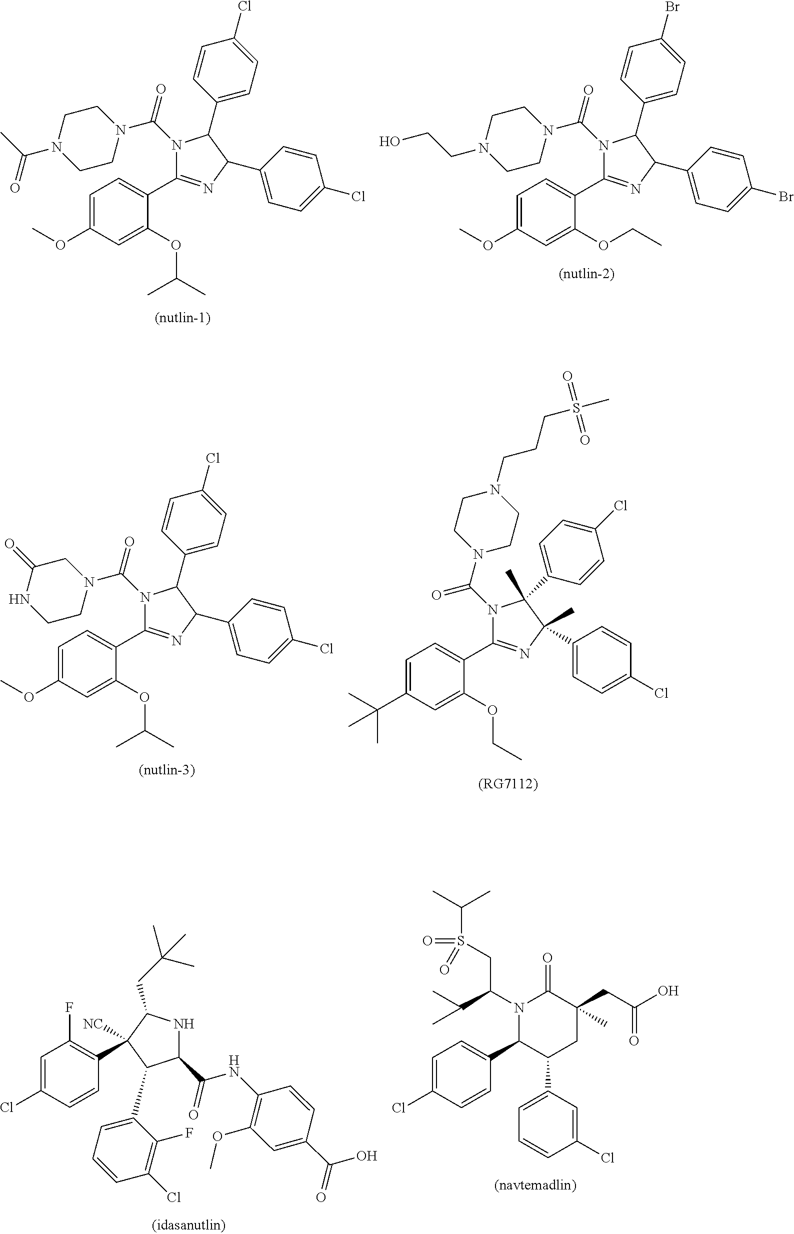

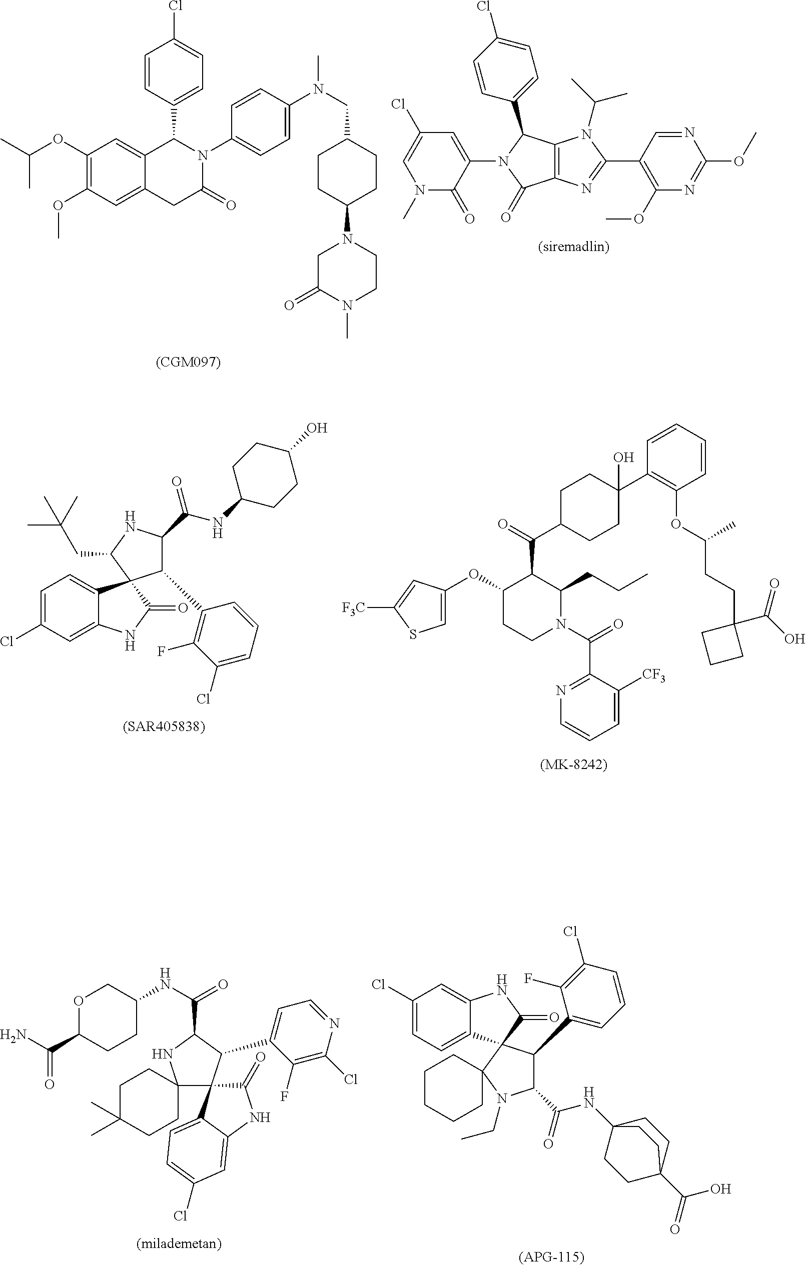

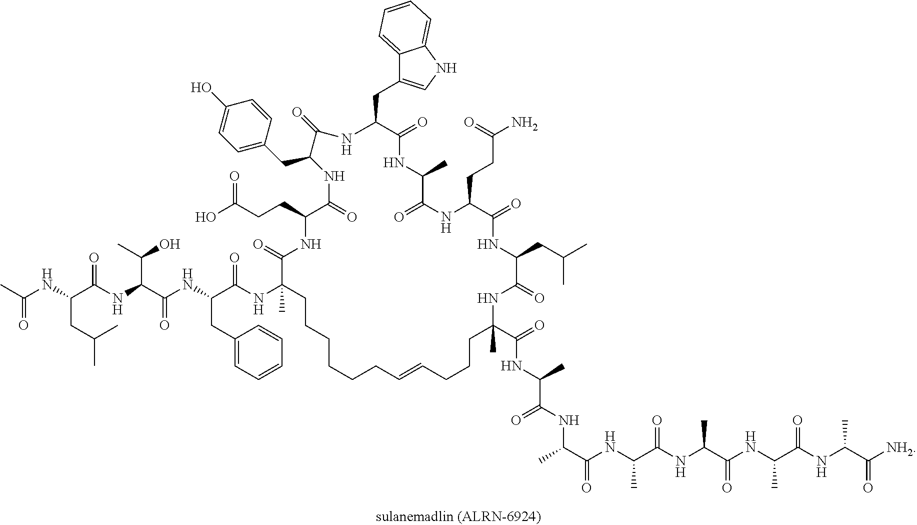 [SMILES: CC(=O)N[C@@H](CC(C)C)C(=O)N[C@H](C(=O)N[C@@H](Cc1ccccc1)C(=O)N[C@]1(C)CCCCCC/C=C/CCC[C@@](C)(C(=O)N[C@@H](C)C(=O)N[C@@H](C)C(=O)N[C@@H](C)C(=O)N[C@@H](C)C(=O)N[C@@H](C)C(=O)N[C@H](C)C(N)=O)NC(=O)[C@H](CC(C)C)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]

In some embodiments, the MDM2 inhibitor is nutlin-1:

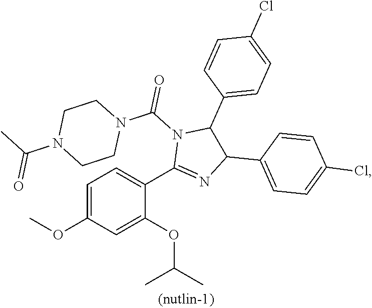 [SMILES: COc1ccc(C2=NC(c3ccc(Cl)cc3)C(c3ccc(Cl)cc3)N2C(=O)N2CCN(C(C)=O)CC2)c(OC(C)C)c1]

or a pharmaceutically acceptable salt thereof.

In some embodiments, the MDM2 inhibitor is nutlin-2:

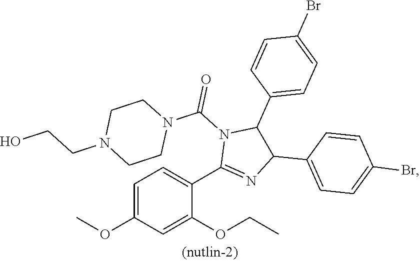 [SMILES: CCOc1cc(OC)ccc1C1=NC(c2ccc(Br)cc2)C(c2ccc(Br)cc2)N1C(=O)N1CCN(CCO)CC1]

or a pharmaceutically acceptable salt thereof.

In some embodiments, the MDM2 inhibitor is nutlin-3:

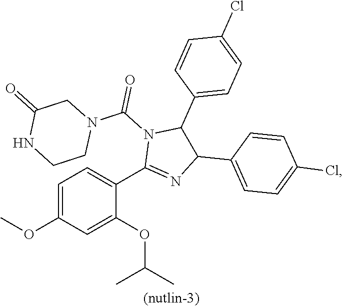 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically acceptable salt thereof.

In some embodiments, the MDM2 inhibitor is RG7112:

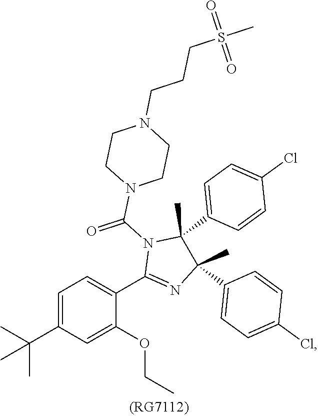 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically acceptable salt thereof.

In some embodiments, the MDM2 inhibitor is idasanutlin:

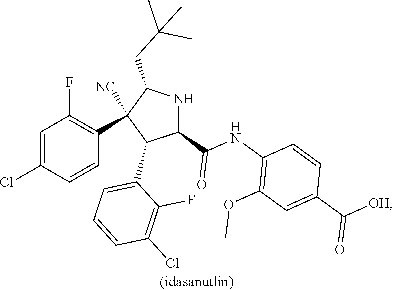

or a pharmaceutically acceptable salt thereof.

In some embodiments, the MDM2 inhibitor is navtemadlin:

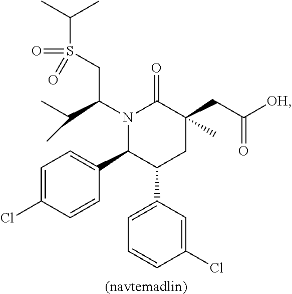

or a pharmaceutically acceptable salt thereof.

In some embodiments, the MDM2 inhibitor is CGM097:

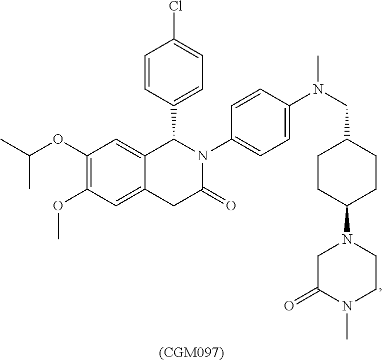

or a pharmaceutically acceptable salt thereof.

In some embodiments, the MDM2 inhibitor is siremadlin:

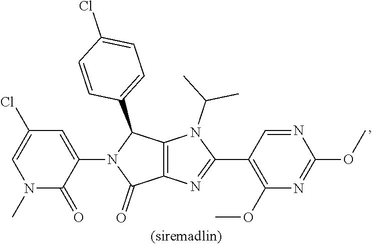 [SMILES: COc1ncc(-c2nc3c(n2C(C)C)[C@H](c2ccc(Cl)cc2)N(c2cc(Cl)cn(C)c2=O)C3=O)c(OC)n1]

or a pharmaceutically acceptable salt thereof.

In some embodiments, the MDM2 inhibitor is SAR405838:

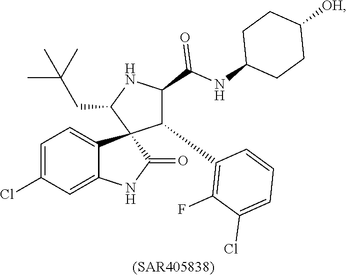

or a pharmaceutically acceptable salt thereof.

In some embodiments, the MDM2 inhibitor is MK-8242:

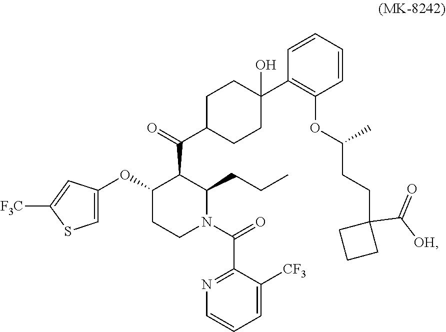 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically acceptable salt thereof.

In some embodiments, the MDM2 inhibitor is milademetan:

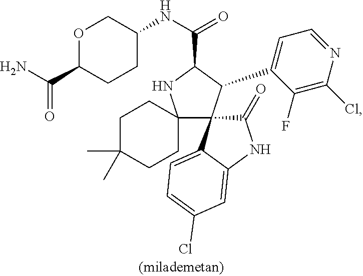 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

or a pharmaceutically acceptable salt thereof.

In some embodiments, the MDM2 inhibitor is APG-115:

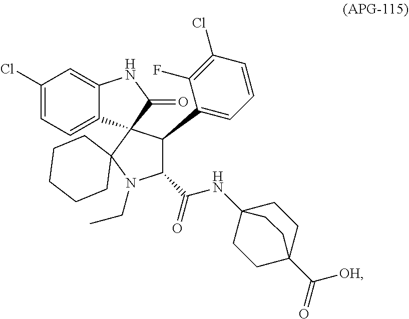 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof.

In some embodiments, the MDM2 inhibitor is nutlin-3a:

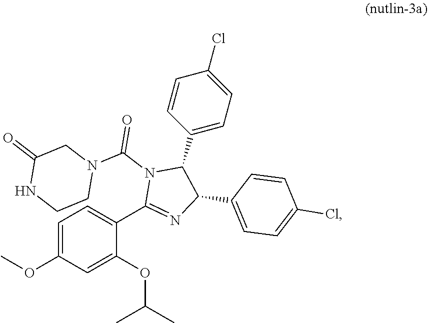 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

or a pharmaceutically-acceptable salt thereof.

In some embodiments, the MDM2 inhibitor is:

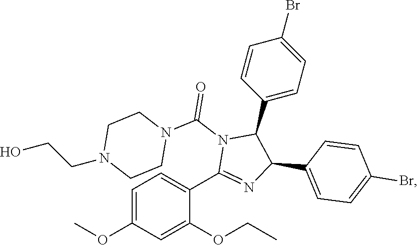 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof.

In some embodiments, the MDM2 inhibitor is:

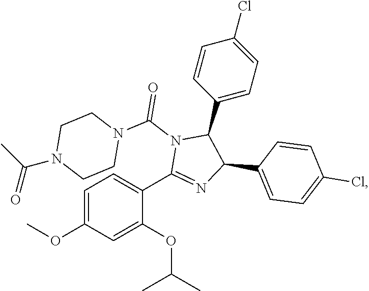 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, pyrazine, or pyrimidine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, an imidazole salt, a pyrazine salt, or a pyrimidine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt , or a maleate salt.

### Pharmaceutical Compositions of the Invention.

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

A pharmaceutical composition of the disclosure can be administered intratumorally. In some embodiments, a pharmaceutical composition for intratumoral injection can be a solution. In some embodiments, a pharmaceutical composition for intratumoral injection can be a controlled-release solution. In some embodiments, a pharmaceutical composition for intratumoral injection can be a gel. In some embodiments, a pharmaceutical composition for intratumoral injection can be a controlled-release gel.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

A pharmaceutical composition of the disclosure can comprise a polymeric agent for controlled release. In some embodiments, the pharmaceutical composition can comprise from about 50 to about 100 mg/dose, from about 100 to about 200 mg/dose, from about 200 to about 300 mg/dose, from about 300 to about 400 mg/dose, or from about 400 to about 500 mg/dose of the polymer for controlled release. In some embodiments, the pharmaceutical composition can comprise about 50 mg/dose, about 100 mg/dose, about 150 mg/dose, about 200 mg/dose, about 250 mg/dose, about 300 mg/dose, about 350 mg/dose, about 400 mg/dose, about 450 mg/dose, or about 500 mg/dose of the polymer for controlled release.

In some embodiments, a pharmaceutical composition of the disclosure can comprise from about 1% to about 99% of a polymeric agent for controlled release. In some embodiments, a pharmaceutical composition of the disclosure can comprise from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 99% (w/w) of a polymeric agent for controlled release. In some embodiments, a pharmaceutical composition of the disclosure can comprise about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% (w/w) of a polymeric agent for controlled release.

In some embodiments, the polymer for controlled release is a biodegradable polymer. In some embodiments, the polymer for controlled release is a polymer that degrades by hydrolysis in water (i.e., hydrolysis or hydrolytic degradation). In some embodiments, the polymer for controlled release is a polymer that degrades in vivo resulting solely from hydrolysis by the water present in tissues and organs. In some embodiments, the polymer for controlled release is formulated as a thermogel.

In some embodiments, the polymer for controlled release is poly(glycolic acid) (PGA), poly(lactic acid) (PLA), or poly(lactic-co-glycolic acid) (PLGA). In some embodiments, the controlled-release polymer is poly(D,L-lactic acid) or poly(D,L-lactic-co-glycolic acid). In some embodiments, the polymer for controlled release is a block copolymer of PLGA and PEG (PLGA-PEG), for example, a PLGA-PEG (AB) diblock or PLGA-PEG-PLGA (ABA) repeating copolymer. In some embodiments, the polymer for controlled release is poly(caprolactone) (PCL). In some embodiments, the polymer for controlled release is a PCL block copolymer comprising PEG, poly(acrylic acid) (PAA), poly(-ethyl-2-oxazoline) (PEtOz), poly(N-isopropylacrylamide) (PNIPAAm), or poly(N,N-dimethylamino-2-ethyl methacrylate) (PDMAEMA) conjugated to a hydrophobic PCL segment. In some embodiments, the polymer for controlled release is poly(alkyl cyanoacrylate) (PACA). In some embodiments, the polymer for controlled release is a deblock copolymer with PEG and PACA blocks, or a triblock copolymer with PEG and PACA blocks. In some embodiments, the polymer for controlled release is a hydroxypropyl)methacrylamide copolymer.

In some embodiments, the polymer for controlled release is a poly(ortho ester) (POE). In some embodiments, the polymer for controlled release is a poly(anhydride). In some embodiments, the polymer for controlled release is a poly(amide) or poly(amino acid), for example, poly(γ-glutamic acid) or poly(L-lysine). In some embodiments, the polymer for controlled release is a poly(ester amide) (PEA) or a poly((β-amino ester) (PBAE). In some embodiments, the polymer for controlled release is a poly(phosphoester).

In some embodiments, the polymer for controlled release is a naturally occurring biodegradable polymer. In some embodiments, the polymer for controlled release is a protein-based polymer, such as collagen, albumin, gelatin; or a polysaccharide, such as agarose, alginate, carrageenan, hyaluronic acid (HA), dextran, chitosan, and cyclodextrin.

In some embodiments, a pharmaceutical composition of the disclosure comprises from about 50 mg/mL to about 750 mg/mL of a polymer for controlled release. In some embodiments, a pharmaceutical composition of the disclosure comprises from about 50 mg/mL to about 100 mg/mL, from about 100 mg/mL to about 150 mg/mL, from about 150 mg/mL to about 200 mg/mL, from about 200 mg/mL to about 250 mg/mL, from about 250 mg/mL to about 300 mg/mL, from about 350 mg/mL to about 400 mg/mL, from about 400 mg/mL to about 450 mg/mL, from about 450 mg/mL to about 500 mg/mL, from about 500 mg/mL to about 550 mg/mL, from about 550 mg/mL to about 600 mg/mL, from about 600 mg/mL to about 650 mg/mL, from about 650 mg/mL to about 700 mg/mL, or from about 700 mg/mL to about 750 mg/mL of a polymer for controlled release. In some embodiments, a pharmaceutical composition of the disclosure comprises about 50 mg/mL, about 100 mg/mL, about 150 mg/mL, about 200 mg/mL, about 250 mg/mL, about 300 mg/mL, about 350 mg/mL, about 400 mg/mL, about 450 mg/mL, about 500 mg/mL, about 550 mg/mL, about 600 mg/mL, about 650 mg/mL, about 700 mg/mL, or about 750 mg/mL of a polymer for controlled release.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a pharmaceutical composition can be prepared at a concentration of about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, about 4.0 mg/mL, about 4.2 mg/mL, about 4.4 mg/mL, about 4.6 mg/mL, about 4.8 mg/mL, or about 5 mg/mL. In some embodiments, a pharmaceutical composition can be prepared at a concentration of about 1 mg/mL. In some embodiments, a pharmaceutical composition can be prepared at a concentration of about 2 mg/mL. In some embodiments, a pharmaceutical composition can be prepared at a concentration of about 3 mg/mL. In some embodiments, a pharmaceutical composition can be prepared at a concentration of about 4 mg/mL. In some embodiments, a pharmaceutical composition can be prepared at a concentration of about 5 mg/mL.

In some embodiments, a dose can be expressed in terms of an amount of the drug divided by the mass of the subject, for example, milligrams of drug per kilograms of subject body mass. In some embodiments, a compound is administered in an amount ranging from about 0.001 mg/kg to about 0.005 mg/kg, from about 0.005 mg/kg to about 0.01 mg/kg, from about 0.01 mg/kg to about 0.015 mg/kg, from about 0.015 mg/kg to about 0.02 mg/kg, from about 0.02 mg/kg to about 0.025 mg/kg, from about 0.025 mg/kg to about 0.03 mg/kg, from about 0.03 mg/kg to about 0.035 mg/kg, from about 0.035 mg/kg to about 0.04 mg/kg, from about 0.04 mg/kg to about 0.045 mg/kg, from about 0.045 mg/kg to about 0.05 mg/kg, from about 0.05 mg/kg to about 0.055 mg/kg, from about 0.055 mg/kg to about 0.06 mg/kg, from about 0.06 mg/kg to about 0.065 mg/kg, from about 0.065 mg/kg to about 0.07 mg/kg, from about 0.07 mg/kg to about 0.075 mg/kg, from about 0.075 mg/kg to about 0.08 mg/kg, from about 0.08 mg/kg to about 0.085 mg/kg, from about 0.085 mg/kg to about 0.09 mg/kg, from about 0.09 mg/kg to about 0.095 mg/kg, from about 0.095 mg/kg to about 0.1 mg/kg. In some embodiments, a compound is administered in an amount ranging from about 0.1 mg/kg to about 0.2 mg/kg, from about 0.2 mg/kg to about 0.3 mg/kg, from about 0.3 mg/kg to about 0.4 mg/kg, from about 0.4 mg/kg to about 0.5 mg/kg, from about 0.5 mg/kg to about 0.6 mg/kg, from about 0.6 mg/kg to about 0.7 mg/kg, from about 0.7 mg/kg to about 0.8 mg/kg, from about 0.8 mg/kg to about 0.9 mg/kg, or from about 0.9 mg/kg to about 1 mg/kg. In some embodiments, a compound is administered in an amount of from about 1 mg/kg to about 5 mg/kg, from about 5 mg/kg to about 50 mg/kg, from about 250 mg/kg to about 2000 mg/kg, from about 10 mg/kg to about 800 mg/kg, from about 50 mg/kg to about 400 mg/kg, from about 100 mg/kg to about 300 mg/kg, or from about 150 mg/kg to about 200 mg/kg.

In some embodiments, a dose of a pharmaceutical composition of the disclosure can comprise from about 0.01 mg to about 0.05 mg, from about 0.05 mg to about 0.1 mg, from about 0.1 mg to about 0.15 mg, from about 0.15 mg to about 0.2 mg, from about 0.2 mg to about 0.25 mg, from about 0.25 mg to about 0.3 mg, from about 0.3 mg to about 0.35 mg, from about 0.35 mg to about 0.4 mg, from about 0.4 mg to about 0.45 mg, from about 0.45 mg to about 0.5 mg, from about 0.5 mg to about 0.6 mg, from about 0.6 mg to about 0.7 mg, from about 0.7 mg to about 0.8 mg, from about 0.8 mg to about 0.9 mg, from about 0.9 mg to about 1 mg, from about 1 mg to about 1.1 mg, from about 1.1 mg to about 1.2 mg, from about 1.2 mg to about 1.3 mg, from about 1.3 mg to about 1.4 mg, from about 1.4 mg to about 1.5 mg, from about 1.5 mg to about 1.6 mg, from about 1.6 mg to about 1.7 mg, from about 1.7 mg to about 1.8 mg, from about 1.8 mg to about 1.9 mg, from about 1.9 mg to about 2 mg, from about 2 mg to about 2.1 mg, from about 2.1 mg to about 2.2 mg, from about 2.2 mg to about 2.3 mg, from about 2.3 mg to about 2.4 mg, from about 2.4 mg to about 2.5 mg, from about 2.5 mg to about 2.6 mg, from about 2.6 mg to about 2.7 mg, from about 2.7 mg to about 2.8 mg, from about 2.8 mg to about 2.9 mg, from about 2.9 mg to about 3 mg, from about 3 mg to about 3.1 mg, from about 3.1 mg to about 3.2 mg, from about 3.2 mg to about 3.3 mg, from about 3.3 mg to about 3.4 mg, from about 3.4 mg to about 3.5 mg, from about 3.5 mg to about 3.6 mg, from about 3.6 mg to about 3.7 mg, from about 3.7 mg to about 3.8 mg, from about 3.8 mg to about 3.9 mg, from about 3.9 mg to about 4 mg, from about 4 mg to about 4.1 mg, from about 4.1 mg to about 4.2 mg, from about 4.2 mg to about 4.3 mg, from about 4.3 mg to about 4.4 mg, from about 4.4 mg to about 4.5 mg, from about 4.5 mg to about 4.6 mg, from about 4.6 mg to about 4.7 mg, from about 4.7 mg to about 4.8 mg, from about 4.8 mg to about 4.9 mg, or from about 4.9 mg to about 5 mg.

In some embodiments, a dose can be expressed in terms of an amount of the drug divided by the volume of a tumor, for example, milligrams of drug per cm³ of a tumor volume. In some embodiments, a dose of a pharmaceutical composition of the disclosure can comprise from about 0.01 mg/cm³ to about 1 mg/cm³. In some embodiments, a dose of a pharmaceutical composition of the disclosure can comprise from about 0.01 mg/cm³ to about 0.02 mg/cm³, from about 0.02 mg/cm³ to about 0.03 mg/cm³, from about 0.03 mg/cm³ to about 0.04 mg/cm³, from about 0.04 mg/cm³ to about 0.05 mg/cm³, from about 0.05 mg/cm³ to about 0.06 mg/cm³, from about 0.06 mg/cm³ to about 0.07 mg/cm³, from about 0.07 mg/cm³ to about 0.08 mg/cm³, from about 0.08 mg/cm³ to about 0.09 mg/cm³, from about 0.09 mg/cm³ to about 0.1 mg/cm³, from about 0.1 mg/cm³ to about 0.2 mg/cm³, from about 0.2 mg/cm³ to about 0.3 mg/cm³, from about 0.3 mg/cm³ to about 0.4 mg/cm³, from about 0.4 mg/cm³ to about 0.5 mg/cm³, from about 0.5 mg/cm³ to about 0.6 mg/cm³, from about 0.6 mg/cm³ to about 0.7 mg/cm³, from about 0.7 mg/cm³ to about 0.8 mg/cm³, from about 0.8 mg/cm³ to about 0.9 mg/cm³, from about 0.9 mg/cm³ to about 1 mg/cm³. In some embodiments, a dose of a pharmaceutical composition of the disclosure can comprise about 0.02 mg/cm³, about 0.04 mg/cm³, about 0.06 mg/cm³, about 0.08 mg/cm³, about 0.1 mg/cm³, about 0.2 mg/cm³, about 0.3 mg/cm³, about 0.4 mg/cm³, about 0.5 mg/cm³, about 0.6 mg/cm³, about 0.7 mg/cm³, about 0.8 mg/cm³, about 0.9 mg/cm³, or 1 mg/cm³.

### Methods of Use

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some embodiments, the cancer or tumor is breast cancer. In some embodiments, the cancer or tumor is ovarian cancer. In some embodiments, the cancer or tumor is pancreatic cancer. In some embodiments, the compounds of the invention show non-lethal toxicity.

In some embodiments, a compound or pharmaceutical composition of the disclosure can be administered intratumorally. In some embodiments, a compound or pharmaceutical composition of the disclosure can be administered intratumorally by pulling a needle through a tumor mass.

In some embodiments, a sterilized needle is used to inject a compound or pharmaceutical composition of the disclosure intratumorally. In some embodiments, a 30G needle can be used to inject a compound or pharmaceutical composition of the disclosure intratumorally. In some embodiments, a 20G needle can be used to inject a compound or pharmaceutical composition of the disclosure intratumorally. In some embodiments, a 18G needle can be used to inject a compound or pharmaceutical composition of the disclosure intratumorally. In some embodiments, a 16G needle can be used to inject a compound or pharmaceutical composition of the disclosure intratumorally. In some embodiments, a 14G needle can be used to inject a compound or pharmaceutical composition of the disclosure intratumorally. In some embodiments, a 12G needle can be used to inject a compound or pharmaceutical composition of the disclosure intratumorally.

In some embodiments, a compound or pharmaceutical composition of the disclosure can be administered intratumorally in a volume of from about 5 μL, to about 50 μL. In some embodiments, a compound or pharmaceutical composition of the disclosure can be administered intratumorally in a volume of from about 5 μLto about 10 μL from about 10 μL to about 15 μL, from about 15 μL to about 20 μL, from about 20 μL to about 25 μL from about 25 μL to about 30 μLfrom about 30 μL to about 35 μL, from about 35 μL to about 40 μL from about 40 μL to about 45 μL, or from about 45 μL, to about 50 μL. In some embodiments, a compound or pharmaceutical composition of the disclosure can be administered intratumorally in a volume of from about 5 μLto about 10 p.L. In some embodiments, a compound or pharmaceutical composition of the disclosure can be administered intratumorally in a volume of from about 10 μL to about 15 μL. In some embodiments, a compound or pharmaceutical composition of the disclosure can be administered intratumorally in a volume of from about 15 μL, to about 20 μL. In some embodiments, a compound or pharmaceutical composition of the disclosure can be administered intratumorally in a volume of from about 20 μL to about 25 μL. In some embodiments, a compound or pharmaceutical composition of the disclosure can be administered intratumorally in a volume of from about 25 μL to about 30 μL.

In some embodiments, a compound or pharmaceutical composition of the disclosure can be administered intratumorally in a volume of from about 5 μL, about 10 μL, about 15 μL, about 20 μL, about 25 μL, about 30 μL, about 35 μL, about 40 μL, about 45 μL, or about 50 μL. In some embodiments, a compound or pharmaceutical composition of the disclosure can be administered intratumorally in a volume of about 10 μL. In some embodiments, a compound or pharmaceutical composition of the disclosure can be administered intratumorally in a volume of about 15 μL. In some embodiments, a compound or pharmaceutical composition of the disclosure can be administered intratumorally in a volume of about 20 μL. In some embodiments, a compound or pharmaceutical composition of the disclosure can be administered intratumorally in a volume of about 25 μL. In some embodiments, a compound or pharmaceutical composition of the disclosure can be administered intratumorally in a volume of about 30 μL.

A compound or pharmaceutical composition of the disclosure can be administered at least once a day (QD), at least once every 3 days (Q3D), at least once every 7 days (Q7D), or at least once every 14 days (Q14D). In some embodiments, a compound or pharmaceutical composition of the disclosure can be administered at least once a day (QD). In some embodiments, a compound or pharmaceutical composition of the disclosure can be administered at least once every 3 days (Q3D). In some embodiments, a compound or pharmaceutical composition of the disclosure can be administered at least once every 7 days (Q7D). In some embodiments, a compound or pharmaceutical composition of the disclosure can be administered at least once every 14 days (Q14D). In some embodiments, a compound or pharmaceutical composition of the disclosure can be administered at least once every month. In some embodiments, a compound or pharmaceutical composition of the disclosure can be administered at least once every 2 months. In some embodiments, a compound or pharmaceutical composition of the disclosure can be administered at least once every 3 months. In some embodiments, a compound or pharmaceutical composition of the disclosure can be administered at least once every 6 months.

A compound or pharmaceutical composition of the disclosure can be administered in at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or at least 31 doses. In some embodiments, a compound or pharmaceutical composition of the disclosure can be administered in at least 3 doses. In some embodiments, a compound or pharmaceutical composition of the disclosure can be administered in at least 5 doses. In some embodiments, a compound or pharmaceutical composition of the disclosure can be administered in at least 9 doses. In some embodiments, a compound or pharmaceutical composition of the disclosure can be administered in at least 12 doses. In some embodiments, a compound or pharmaceutical composition of the disclosure can be administered in at least 31 doses.

In some embodiments, a compound or pharmaceutical composition of the disclosure can be administered once a day for 31 days (QD×31). In some embodiments, a compound or pharmaceutical composition of the disclosure can be administered once every 3 days for a total of 9 doses (Q3D×9). In some embodiments, a compound or pharmaceutical composition of the disclosure can be administered once every 7 days for a total of 5 doses (Q7D×5). In some embodiments, a compound or pharmaceutical composition of the disclosure can be administered once every 14 days for a total of 3 doses (Q14D×3).

In some embodiments, the therapeutically-effective amount of the compound is from about 0.001 mg/kg to about 0.005 mg/kg, from about 0.005 mg/kg to about 0.01 mg/kg, from about 0.01 mg/kg to about 0.015 mg/kg, from about 0.015 mg/kg to about 0.02 mg/kg, from about 0.02 mg/kg to about 0.025 mg/kg, from about 0.025 mg/kg to about 0.03 mg/kg, from about 0.03 mg/kg to about 0.035 mg/kg, from about 0.035 mg/kg to about 0.04 mg/kg, from about 0.04 mg/kg to about 0.045 mg/kg, from about 0.045 mg/kg to about 0.05 mg/kg, from about 0.05 mg/kg to about 0.055 mg/kg, from about 0.055 mg/kg to about 0.06 mg/kg, from about 0.06 mg/kg to about 0.065 mg/kg, from about 0.065 mg/kg to about 0.07 mg/kg, from about 0.075 mg/kg to about 0.08 mg/kg, from about 0.08 mg/kg to about 0.085 mg/kg, from about 0.085 mg/kg to about 0.09 mg/kg, from about 0.09 mg/kg to about 0.095 mg/kg, or from about 0.095 mg/kg to about 0.1 mg/kg. In some embodiments, the therapeutically-effective amount is from about 0.1 mg/kg to about 0.2 mg/kg, from about 0.2 mg/kg to about 0.3 mg/kg, from about 0.3 mg/kg to about 0.4 mg/kg, from about 0.4 mg/kg to about 0.5 mg/kg, from about 0.5 mg/kg to about 0.6 mg/kg, from about 0.6 mg/kg to about 0.7 mg/kg, from about 0.7 mg/kg to about 0.8 mg/kg, from about 0.8 mg/kg to about 0.9 mg/kg, or from about 0.9 mg/kg to about 1 mg/kg. In some embodiments, the therapeutically-effective amount is from about 1 mg/kg to about 5 mg/kg, from about 5 mg/kg to about 50 mg/kg, from about 250 mg/kg to about 2000 mg/kg, from about 10 mg/kg to about 800 mg/kg, from about 50 mg/kg to about 400 mg/kg, from about 100 mg/kg to about 300 mg/kg, or from about 150 mg/kg to about 200 mg/kg.

In some embodiments, the therapeutically-effective amount is from about 0.01 mg to about 0.05 mg, from about 0.05 mg to about 0.1 mg, from about 0.1 mg to about 0.15 mg, from about 0.15 mg to about 0.2 mg, from about 0.2 mg to about 0.25 mg, from about 0.25 mg to about 0.3 mg, from about 0.3 mg to about 0.35 mg, from about 0.35 mg to about 0.4 mg, from about 0.4 mg to about 0.45 mg, from about 0.45 mg to about 0.5 mg, from about 0.5 mg to about 0.6 mg, from about 0.6 mg to about 0.7 mg, from about 0.7 mg to about 0.8 mg, from about 0.8 mg to about 0.9 mg, from about 0.9 mg to about 1 mg, from about 1 mg to about 1.1 mg, from about 1.1 mg to about 1.2 mg, from about 1.2 mg to about 1.3 mg, from about 1.3 mg to about 1.4 mg, from about 1.4 mg to about 1.5 mg, from about 1.5 mg to about 1.6 mg, from about 1.6 mg to about 1.7 mg, from about 1.7 mg to about 1.8 mg, from about 1.8 mg to about 1.9 mg, from about 1.9 mg to about 2 mg, from about 2 mg to about 2.1 mg, from about 2.1 mg to about 2.2 mg, from about 2.2 mg to about 2.3 mg, from about 2.3 mg to about 2.4 mg, from about 2.4 mg to about 2.5 mg, from about 2.5 mg to about 2.6 mg, from about 2.6 mg to about 2.7 mg, from about 2.7 mg to about 2.8 mg, from about 2.8 mg to about 2.9 mg, from about 2.9 mg to about 3 mg, from about 3 mg to about 3.1 mg, from about 3.1 mg to about 3.2 mg, from about 3.2 mg to about 3.3 mg, from about 3.3 mg to about 3.4 mg, from about 3.4 mg to about 3.5 mg, from about 3.5 mg to about 3.6 mg, from about 3.6 mg to about 3.7 mg, from about 3.7 mg to about 3.8 mg, from about 3.8 mg to about 3.9 mg, from about 3.9 mg to about 4 mg, from about 4 mg to about 4.1 mg, from about 4.1 mg to about 4.2 mg, from about 4.2 mg to about 4.3 mg, from about 4.3 mg to about 4.4 mg, from about 4.4 mg to about 4.5 mg, from about 4.5 mg to about 4.6 mg, from about 4.6 mg to about 4.7 mg, from about 4.7 mg to about 4.8 mg, from about 4.8 mg to about 4.9 mg, or from about 4.9 mg to about 5 mg.

In some embodiments, the therapeutically-effective amount is from about 0.01 mg/cm³ to about 0.02 mg/cm³, from about 0.02 mg/cm³ to about 0.03 mg/cm³, from about 0.03 mg/cm³ to about 0.04 mg/cm³, from about 0.04 mg/cm³ to about 0.05 mg/cm³, from about 0.05 mg/cm³ to about 0.06 mg/cm³, from about 0.06 mg/cm³ to about 0.07 mg/cm³, from about 0.07 mg/cm³ to about 0.08 mg/cm³, from about 0.08 mg/cm³ to about 0.09 mg/cm³, from about 0.09 mg/cm³ to about 0.1 mg/cm³, from about 0.1 mg/cm³ to about 0.2 mg/cm³, from about 0.2 mg/cm³ to about 0.3 mg/cm³, from about 0.3 mg/cm³ to about 0.4 mg/cm³, from about 0.4 mg/cm³ to about 0.5 mg/cm³, from about 0.5 mg/cm³ to about 0.6 mg/cm³, from about 0.6 mg/cm³ to about 0.7 mg/cm³, from about 0.7 mg/cm³ to about 0.8 mg/cm³, from about 0.8 mg/cm³ to about 0.9 mg/cm³, or from about 0.9 mg/cm³ to about 1 mg/cm³. In some embodiments, the therapeutically-effective amount is about 0.02 mg/cm³, about 0.04 mg/cm³, about 0.06 mg/cm³, about 0.08 mg/cm³, about 0.1 mg/cm³, about 0.2 mg/cm³, about 0.3 mg/cm³, about 0.4 mg/cm³, about 0.5 mg/cm³, about 0.6 mg/cm³, about 0.7 mg/cm³, about 0.8 mg/cm³, about 0.9 mg/cm³, or 1 mg/cm³.

In some embodiments, administering a compound to a tumor can decrease tumor volume by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 97%. In some embodiments, administering a compound to a tumor can decrease tumor volume by at least about 10%. In some embodiments, administering a compound to a tumor can decrease tumor volume by at least about 30%. In some embodiments, administering a compound to a tumor can decrease tumor volume by at least about 50%.

In some embodiments, the methods of the disclosure further comprise administering a therapeutically-effective amount of a therapeutic agent. In some embodiments, the therapeutic is an immune checkpoint inhibitor, for example, an anti-PD-1 agent or anti-PD-L1 agent. In some embodiments, the anti-PD-1 agent is nivolumab. In some embodiments, the anti-PD-1 agent is pembrolizumab. In some embodiments, the anti-PD-1 agent is cemiplimab. In some embodiments, the anti-PD-L1 agent is atezolizumab. In some embodiments, the anti-PD-L1 agent is avelumab. In some embodiments, the anti-PD-L1 agent is durvalumab.

### Numbered Embodiments

Embodiment 1. A method of treating a tumor in a subject in need thereof comprising administering to the subject a therapeutically-effective amount of a MDM2 inhibitor, wherein the administering is by intratumoral injection at most once every 3 days.

Embodiment 2. The method of embodiment 1, wherein the tumor is breast cancer.

Embodiment 3. The method of embodiment 1, wherein the tumor is ovarian cancer.

Embodiment 4. The method of embodiment 1, wherein the tumor is pancreatic cancer.

Embodiment 5. The method of embodiment 1, wherein the tumor is lung cancer.

Embodiment 6. The method of any one of embodiments 1-5, wherein the administering by intratumoral injection occurs once every 3 days.

Embodiment 7. The method of any one of embodiments 1-5, wherein the administering is by intratumoral injection occurs once every 7 days.

Embodiment 8. The method of any one of embodiments 1-5, wherein the administering is by intratumoral injection occurs once every 14 days.

Embodiment 9. The method of any one of embodiments 1-5, wherein the administering by intratumoral injection occurs once a month.

Embodiment 10. The method of any one of embodiments 1-5, wherein the administering by intratumoral injection occurs once every two months.

Embodiment 11. The method of any one of embodiments 1-10, wherein the administering reduces a tumor volume by at least about 20%.

Embodiment 12. The method of any one of embodiments 1-10, wherein the administering reduces a tumor volume by at least about 50%.

Embodiment 13. The method of any one of embodiments 1-12, wherein the therapeutically-effective amount is from about 0.05 mg to about 50 mg.

Embodiment 14. The method of any one of embodiments 1-12, wherein the therapeutically-effective amount is about 5 mg.

Embodiment 15. The method of any one of embodiments 1-12, wherein the therapeutically-effective amount is about 25 mg.

Embodiment 16. The method of any one of embodiments 1-15, wherein the subject is human.

Embodiment 17. The method of any one of embodiments 1-16, wherein the MDM2 inhibitor is a small molecule.

Embodiment 18. The method of any one of embodiments 1-17, wherein the MDM2 inhibitor is not a linear polyribonucleotide.

Embodiment 19. The method of any one of embodiments 1-18, wherein the MDM2 inhibitor is not an antibody.

Embodiment 20. The method of any one of embodiments 1-19, wherein the MDM2 inhibitor is:

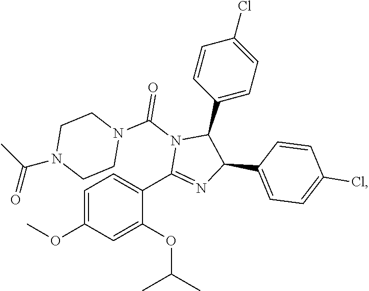 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof.

Embodiment 21. The method of any one of embodiments 1-19, wherein the MDM2 inhibitor is:

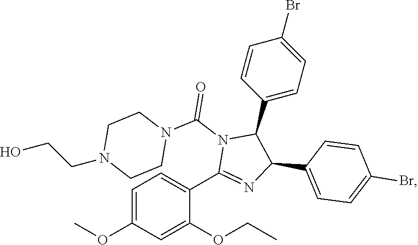 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof.

Embodiment 22. The method of any one of embodiments 1-19, wherein the MDM2 inhibitor is:

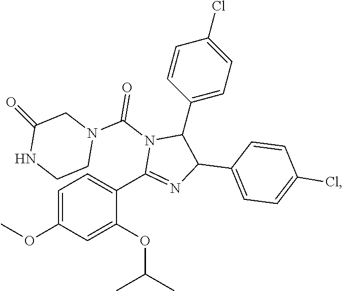 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically-acceptable salt thereof.

Embodiment 23. The method of any one of embodiments 1-19, wherein the MDM2

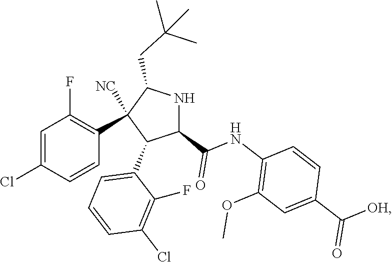 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

or a pharmaceutically-acceptable salt thereof.

Embodiment 24. The method of any one of embodiments 1-19, wherein the MDM2 inhibitor is:

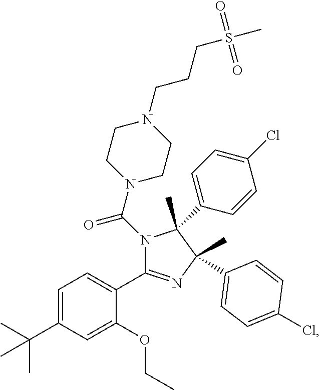 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically-acceptable salt thereof.

Embodiment 25. The method of any one of embodiments 1-19, wherein the MDM2 inhibitor is:

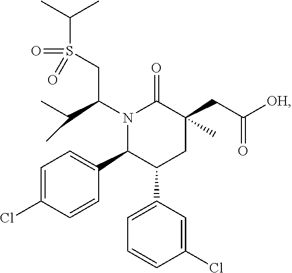 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically-acceptable salt thereof.

Embodiment 26. The method of any one of embodiments 1-19, wherein the MDM2

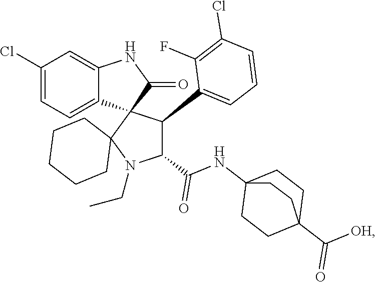 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically-acceptable salt thereof.

Embodiment 27. The method of any one of embodiments 1-19, wherein the MDM2 inhibitor is:

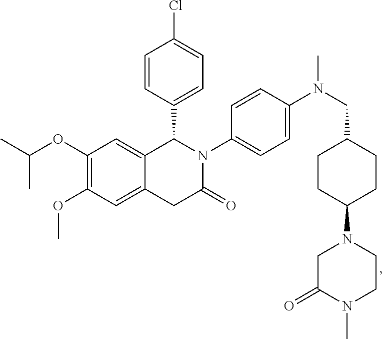

or a pharmaceutically-acceptable salt thereof.

Embodiment 28. The method of any one of embodiments 1-19, wherein the MDM2 inhibitor is:

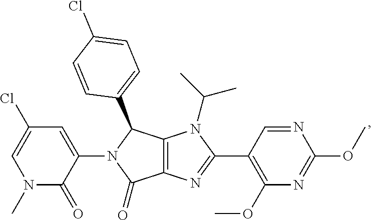 [SMILES: COc1ncc(-c2nc3c(n2C(C)C)[C@H](c2ccc(Cl)cc2)N(c2cc(Cl)cn(C)c2=O)C3=O)c(OC)n1]

or a pharmaceutically-acceptable salt thereof.

Embodiment 29. The method of any one of embodiments 1-19, wherein the MDM2

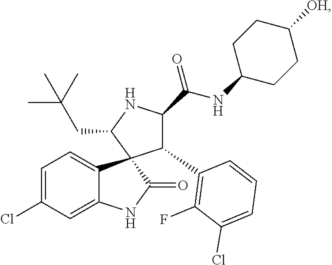 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

or a pharmaceutically-acceptable salt thereof.

Embodiment 30. The method of any one of embodiments 1-19, wherein the MDM2 inhibitor is:

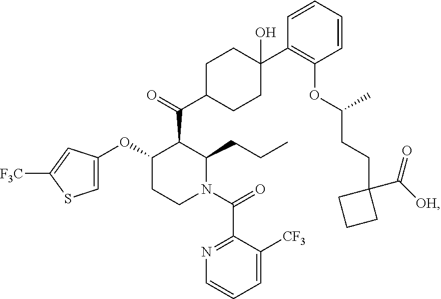 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically-acceptable salt thereof.

Embodiment 31. The method of any one of embodiments 1-19, wherein the MDM2 inhibitor is

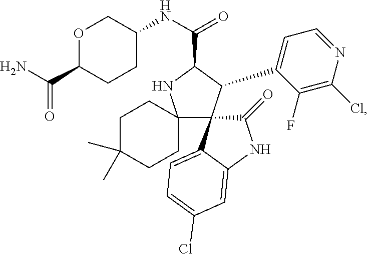 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

or a pharmaceutically-acceptable salt thereof.

Embodiment 32. The method of any one of embodiments 1-19, wherein the MDM2

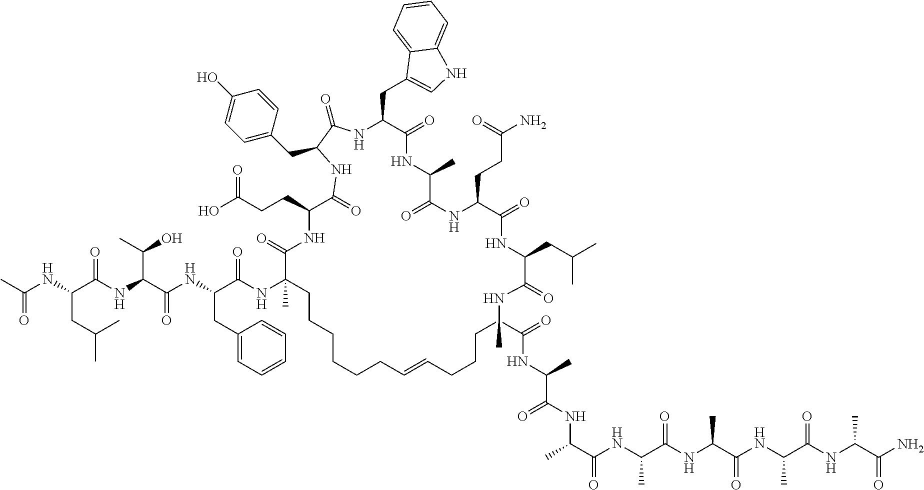 [SMILES: CNC(=O)[C@H](CC(C)C)NC(=O)[C@H](CCC(N)=O)NC(=O)[C@H](C)NC(=O)[C@H](Cc1c[nH]c2ccccc12)NC(=O)[C@H](Cc1ccc(O)cc1)NC(=O)[C@H](CCC(=O)O)NC(=O)[C@@](C)(CCCCCC/C=C/CCC[C@@H](C)C(=O)N[C@@H](C)C(=O)N[C@@H](C)C(=O)N[C@@H](C)C(=O)N[C@@H](C)C(=O)N[C@@H](C)C(=O)N[C@H](C)C(N)=O)NC(=O)[C@H](Cc1ccccc1)NC(=O)[C@@H](NC(=O)[C@H](CC(C)C)NC(C)=O)[C@@H](C)O]

(SEQ ID NO: 1), or a pharmaceutically-acceptable salt thereof.

Embodiment 33. The method of any one of embodiments 1-32, further comprising administering to the subject a therapeutically-effective amount of an additional therapeutic agent.

Embodiment 34. The method of embodiment 33, wherein the additional therapeutic agent is an immune checkpoint inhibitor.

Embodiment 35. The method of embodiment 34, wherein the immune checkpoint inhibitor is an anti-PD-1 agent.

Embodiment 36. The method of embodiment 35, wherein the anti-PD-1 agent is nivolumab.

Embodiment 37. The method of embodiment 35, wherein the anti-PD-1 agent is pembrolizumab.

Embodiment 38. The method of embodiment 35, wherein the anti-PD-1 agent is cemiplimab.

Embodiment 39. The method of embodiment 34, wherein the immune checkpoint inhibitor is an anti-PD-L1 agent.

Embodiment 40. The method of embodiment 39, wherein the anti-PD-L1 agent is atezolizumab.

Embodiment 41. The method of embodiment 39, wherein the anti-PD-L1 agent is avelumab.

Embodiment 42. The method of embodiment 39, wherein the anti-PD-L1 agent is durvalumab.

Embodiment 43. A method of treating a tumor in a subject in need thereof comprising administering to the subject a pharmaceutical composition, wherein the pharmaceutical composition comprises:
a) a therapeutically-effective amount of a MDM2 inhibitor; and
b) a polymer that degrades by hydrolysis in water,
   wherein the administering is by intratumoral injection.

Embodiment 44. The method of embodiment 43, wherein the tumor is breast cancer.

Embodiment 45. The method of embodiment 43, wherein the tumor is ovarian cancer.

Embodiment 46. The method of embodiment 43, wherein the tumor is pancreatic cancer.

Embodiment 47. The method of embodiment 43, wherein the tumor is lung cancer.

Embodiment 48. The method of any one of embodiments 43-47, wherein the administering by intratumoral injection occurs once every 3 days.

Embodiment 49. The method of any one of embodiments 43-47, wherein the administering is by intratumoral injection occurs once every 7 days.

Embodiment 50. The method of any one of embodiments 43-47, wherein the administering is by intratumoral injection occurs once every 14 days.

Embodiment 51. The method of any one of embodiments 43-47, wherein the administering by intratumoral injection occurs once a month.

Embodiment 52. The method of any one of embodiments 43-47, wherein the administering by intratumoral injection occurs once every two months.

Embodiment 53. The method of any one of embodiments 43-52, wherein the administering reduces a tumor volume by at least about 20%.

Embodiment 54. The method of any one of embodiments 43-52, wherein the administering reduces a tumor volume by at least about 50%.

Embodiment 55. The method of any one of embodiments 43-54, wherein the therapeutically-effective amount is from about 0.05 mg to about 50 mg.

Embodiment 56. The method of any one of embodiments 43-54, wherein the therapeutically-effective amount is about 5 mg.

Embodiment 57. The method of any one of embodiments 43-54, wherein the therapeutically-effective amount is about 25 mg.

Embodiment 58. The method of any one of embodiments 43-57, wherein the subject is human.

Embodiment 59. The method of any one of embodiments 43-58, wherein the MDM2 inhibitor is a compound.

Embodiment 60. The method of any one of embodiments 43-58, wherein the MDM2 inhibitor is not a linear polyribonucleotide.

Embodiment 61. The method of any one of embodiments 43-58, wherein the MDM2 inhibitor is not an antibody.

Embodiment 62. The method of any one of embodiments 43-61, wherein the MDM2 inhibitor is:

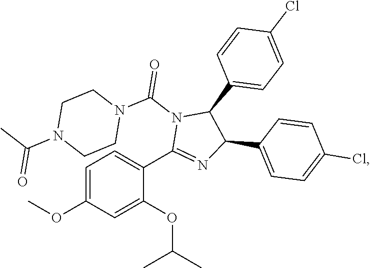 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof.

Embodiment 63. The method of any one of embodiments 43-61, wherein the MDM2 inhibitor is:

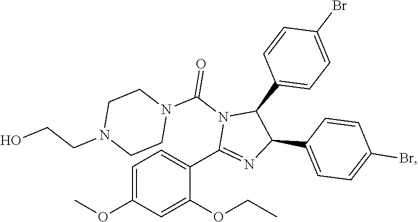 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof.

Embodiment 64. The method of any one of embodiments 43-61, wherein the MDM2

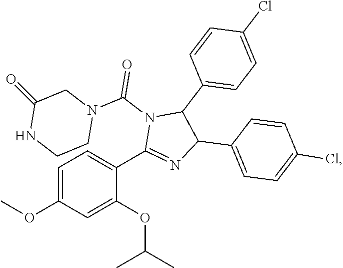 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically-acceptable salt thereof.

Embodiment 65. The method of any one of embodiments 43-61, wherein the MDM2 inhibitor is:

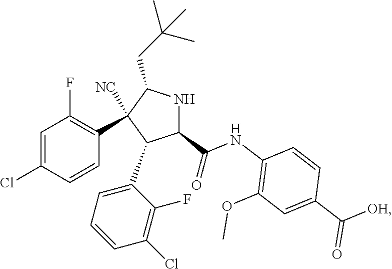 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

or a pharmaceutically-acceptable salt thereof.

Embodiment 66. The method of any one of embodiments 43-61, wherein the MDM2 inhibitor is:

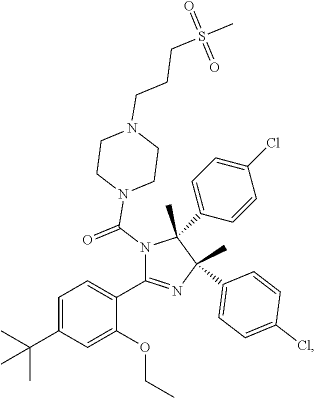 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically-acceptable salt thereof.

Embodiment 67. The method of any one of embodiments 43-61, wherein the MDM2

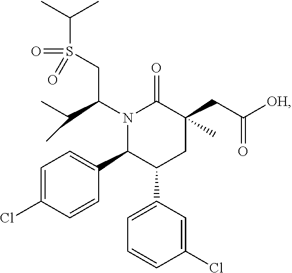 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically-acceptable salt thereof.

Embodiment 68. The method of any one of embodiments 43-61, wherein the MDM2 inhibitor is:

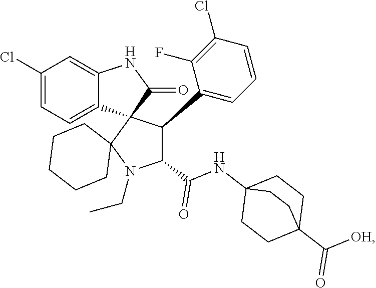 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically-acceptable salt thereof.

Embodiment 69. The method of any one of embodiments 43-61, wherein the MDM2 inhibitor is:

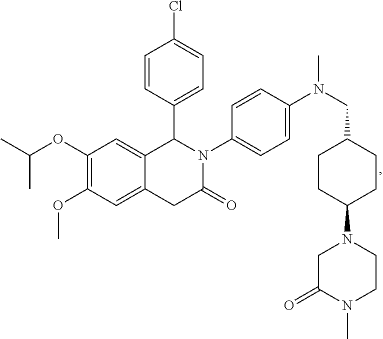

or a pharmaceutically-acceptable salt thereof.

Embodiment 70. The method of any one of embodiments 43-61, wherein the MDM2

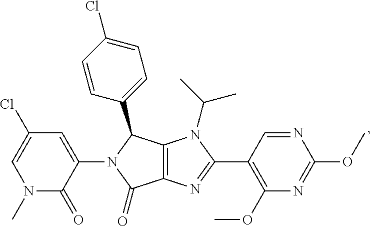 [SMILES: COc1ncc(-c2nc3c(n2C(C)C)[C@H](c2ccc(Cl)cc2)N(c2cc(Cl)cn(C)c2=O)C3=O)c(OC)n1]

or a pharmaceutically-acceptable salt thereof.

Embodiment 71. The method of any one of embodiments 43-61, wherein the MDM2 inhibitor is:

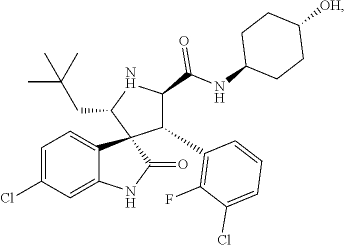 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

or a pharmaceutically-acceptable salt thereof.

Embodiment 72. The method of any one of embodiments 43-61, wherein the MDM2 inhibitor is:

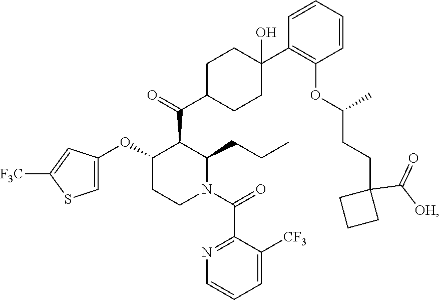 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically-acceptable salt thereof.

Embodiment 73. The method of any one of embodiments 43-61, wherein the MDM2

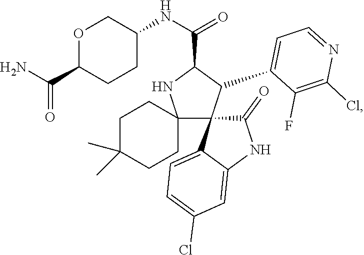

or a pharmaceutically-acceptable salt thereof.

Embodiment 74. The method of any one of embodiments 43-61, wherein the MDM2 inhibitor is:

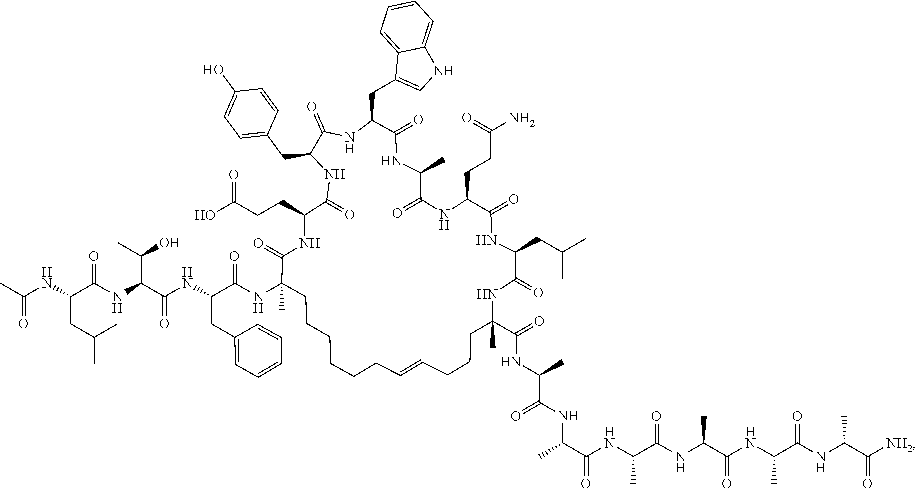 [SMILES: CC(=O)N[C@@H](CC(C)C)C(=O)N[C@H](C(=O)N[C@@H](Cc1ccccc1)C(=O)N[C@]1(C)CCCCCC/C=C/CCC[C@@](C)(C(=O)N[C@@H](C)C(=O)N[C@@H](C)C(=O)N[C@@H](C)C(=O)N[C@@H](C)C(=O)N[C@@H](C)C(=O)N[C@H](C)C(N)=O)NC(=O)[C@H](CC(C)C)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]

(SEQ ID NO: 1), or a pharmaceutically-acceptable salt thereof

Embodiment 75. The method of any one of embodiments 43-74, further comprising administering to the subject a therapeutically-effective amount of an additional therapeutic agent.

Embodiment 76. The method of embodiment 75, wherein the additional therapeutic agent is an immune checkpoint inhibitor.

Embodiment 77. The method of embodiment 76, wherein the immune checkpoint inhibitor is an anti-PD-1 agent.

Embodiment 78. The method of embodiment 77, wherein the anti-PD-1 agent is nivolumab.

Embodiment 79. The method of embodiment 77, wherein the anti-PD-1 agent is pembrolizumab.

Embodiment 80. The method of embodiment 77, wherein the anti-PD-1 agent is cemiplimab.

Embodiment 81. The method of embodiment 76, wherein the immune checkpoint inhibitor is an anti-PD-L1 agent.

Embodiment 82. The method of embodiment 81, wherein the anti-PD-L1 agent is atezolizumab.

Embodiment 83. The method of embodiment 81, wherein the anti-PD-L1 agent is avelumab.

Embodiment 84. The method of embodiment 81, wherein the anti-PD-L1 agent is durvalumab.

Embodiment 85. The method of any one of embodiments 43-84, wherein the pharmaceutical composition is formulated for controlled release delivery.

Embodiment 86. The method of any one of embodiments 43-85, wherein the polymer is poly(lactic-co-glycolic acid) polymer.

Embodiment 87. The method of any one of embodiments 43-85, wherein the polymer is poly(lactic acid) polymer.

Embodiment 88. The method of any one of embodiments 43-87, wherein the polymer that degrades by hydrolysis in water is from about 20% to about 80% (w/w) of the pharmaceutical composition.

Embodiment 89. The method of any one of embodiments 43-87, wherein the polymer that degrades by hydrolysis in water is about 20% of the pharmaceutical composition.

Embodiment 90. The method of any one of embodiments 43-87, wherein the polymer that degrades by hydrolysis in water is about 50% of the pharmaceutical composition.

Embodiment 91. The method of any one of embodiments 43-87, wherein the pharmaceutical composition further comprises a pharmaceutically-acceptable excipient.

Embodiment 92. A pharmaceutical composition comprising in a unit dosage form:
a) a polymer that degrades by hydrolysis in water; and
b) a therapeutically-effective amount of a MDM2 inhibitor,
   wherein the pharmaceutical composition is formulated for intratumoral injection.

Embodiment 93. The pharmaceutical composition of embodiment 92, wherein the pharmaceutical composition is formulated for controlled release.

Embodiment 94. The pharmaceutical composition of embodiment 92 or 93, wherein the polymer is poly(lactic-co-glycolic acid) polymer.

Embodiment 95. The pharmaceutical composition of any one of embodiments 92-94, wherein the polymer that degrades by hydrolysis in water is from about 20% to about 80% (w/w) of the pharmaceutical composition.

Embodiment 96. The pharmaceutical composition of any one of embodiments 92-94, wherein the polymer that degrades by hydrolysis in water is about 20% (w/w) of the pharmaceutical composition.

Embodiment 97. The pharmaceutical composition of any one of embodiments 92-94, wherein the polymer that degrades by hydrolysis in water is about 50% (w/w) of the pharmaceutical composition.

Embodiment 98. The pharmaceutical composition of any one of embodiments 92-97, further comprising a pharmaceutically-acceptable excipient.

Embodiment 99. The pharmaceutical composition of any one of embodiments 92-98, further comprising a pharmaceutically-acceptable diluent.

Embodiment 100. The pharmaceutical composition of any one of embodiments 92-99, wherein the pharmaceutical composition is formulated for intratumoral administration.

Embodiment 101. The pharmaceutical composition of any one of embodiments 92-100, wherein the therapeutically-effective amount is from about 0.05 mg to about 50 mg.

Embodiment 102. The pharmaceutical composition of any one of embodiments 92-100, wherein the therapeutically-effective amount is about 5 mg.

Embodiment 103. The pharmaceutical composition of any one of embodiments 92-100, wherein the therapeutically-effective amount is about 25 mg.

Embodiment 104. The pharmaceutical composition of any one of embodiments 92-103, wherein the MDM2 inhibitor is:

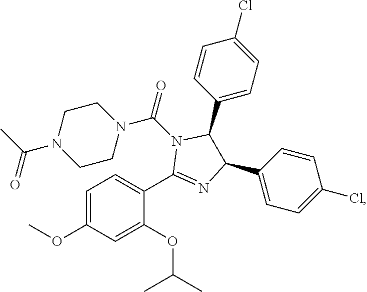 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof.

Embodiment 105. The pharmaceutical composition of any one of embodiments 92-103, wherein the MDM2 inhibitor is:

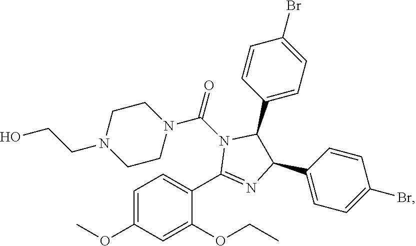 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof.

Embodiment 106. The pharmaceutical composition of any one of embodiments 92-103, wherein the MDM2 inhibitor is:

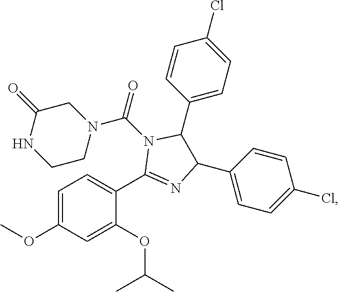 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically-acceptable salt thereof.

Embodiment 107. The pharmaceutical composition of any one of embodiments 92-103, wherein the MDM2 inhibitor is:

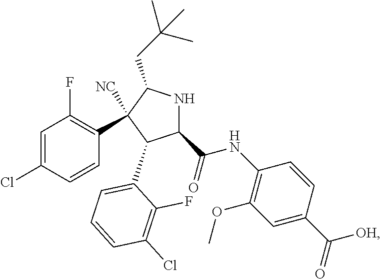 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

or a pharmaceutically-acceptable salt thereof.

Embodiment 108. The pharmaceutical composition of any one of embodiments 92-103, wherein the MDM2 inhibitor is:

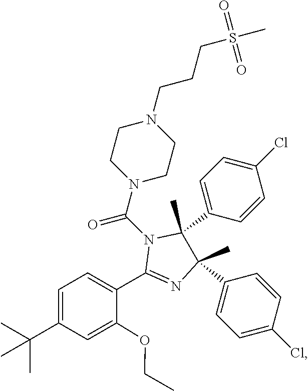 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically-acceptable salt thereof.

Embodiment 109. The pharmaceutical composition of any one of embodiments 92-103, wherein the MDM2 inhibitor is:

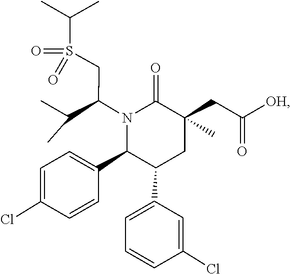 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically-acceptable salt thereof.

Embodiment 110. The pharmaceutical composition of any one of embodiments 92-103, wherein the MDM2 inhibitor is:

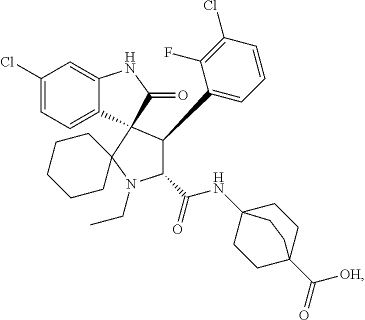 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically-acceptable salt thereof.

Embodiment 111. The pharmaceutical composition of any one of embodiments 92-103, wherein the MDM2 inhibitor is:

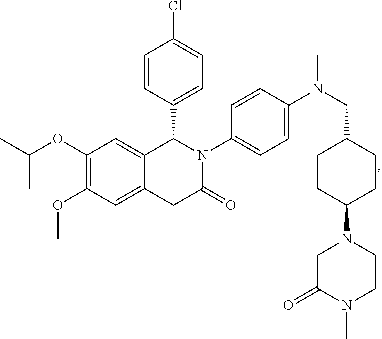

or a pharmaceutically-acceptable salt thereof.

Embodiment 112. The pharmaceutical composition of any one of embodiments 92-103, wherein the MDM2 inhibitor is:

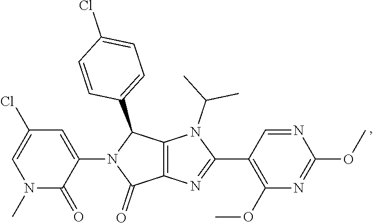

or a pharmaceutically-acceptable salt thereof.

Embodiment 113. The pharmaceutical composition of any one of embodiments 92-103, wherein the MDM2 inhibitor is:

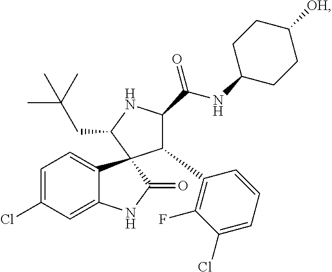 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

or a pharmaceutically-acceptable salt thereof.

Embodiment 114. The pharmaceutical composition of any one of embodiments 92-103, wherein the MDM2 inhibitor is:

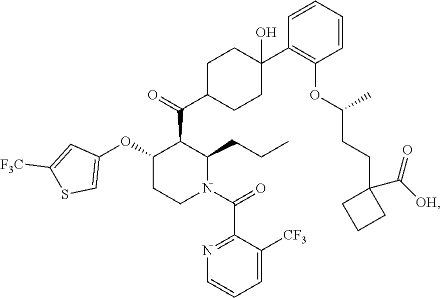 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically-acceptable salt thereof.

Embodiment 115. The pharmaceutical composition of any one of embodiments 92-103, wherein the MDM2 inhibitor is:

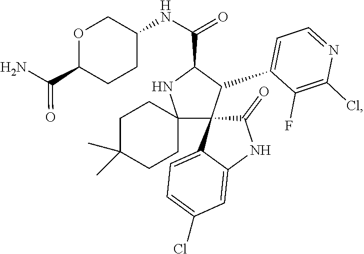

or a pharmaceutically-acceptable salt thereof.

Embodiment 116. The method of any one of embodiments 92-103, wherein the MDM2

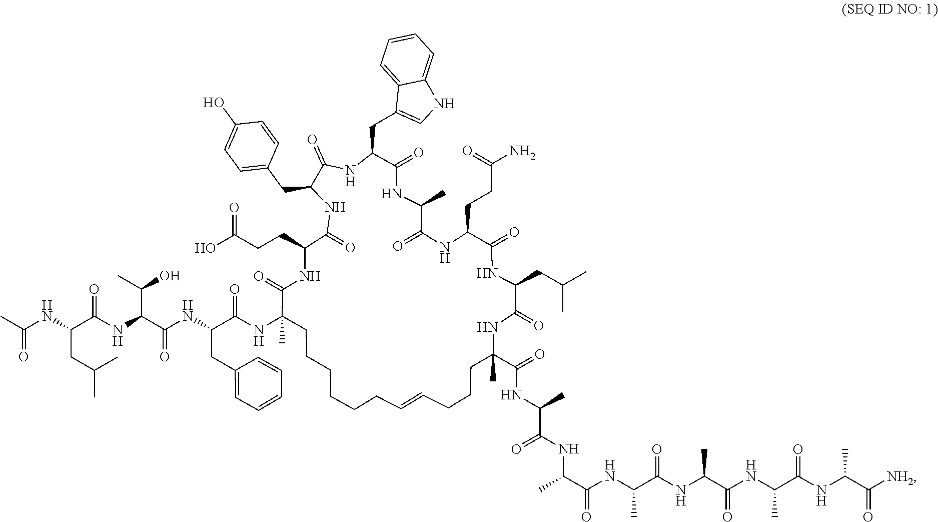 [SMILES: CC(=O)N[C@@H](CC(C)C)C(=O)N[C@H](C(=O)N[C@@H](Cc1ccccc1)C(=O)N[C@]1(C)CCCCCC/C=C/CCC[C@@](C)(C(=O)N[C@@H](C)C(=O)N[C@@H](C)C(=O)N[C@@H](C)C(=O)N[C@@H](C)C(=O)N[C@@H](C)C(=O)N[C@H](C)C(N)=O)NC(=O)[C@H](CC(C)C)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]

or a pharmaceutically-acceptable salt thereof.

Embodiment 117. The pharmaceutical composition of any one of embodiments 92-116, wherein the pharmaceutical composition is a thermogel.

### EXAMPLES

### Example 1: Intratumoral injection of Compounds of the Disclosure

Optimization of intratumoral delivery was performed using blue dye and NUGC3 tumors. NUGC-3 is a human gastric adenocarcinoma cancer cell line. Variables including size of needle, frequency of injection, volume of pharmaceutical composition, and injection technique were examined. Injections frequencies ranged from daily to weekly. A 30 G needle and 15-20 μL volume produced best delivery to tumors of the parameters tried. Needles were pulled through the tumor mass.

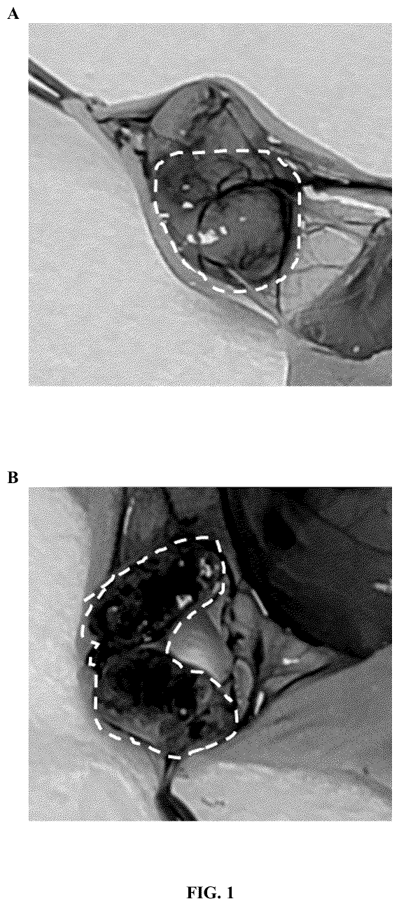

FIG. 1 panels A and B show images of NUGC3 tumors injected intratumorally with blue dye. The dotted lines indicate the tumor mass that turned blue from the dye.

### Example 2: Dosing Regimen Delivered by Intratumoral Administration

A study was conducted to compare differences between dosing regimens of navtemadlin administered to SJSA1 tumors. S.ISa-1 is a human synovial sarcoma cell line. Navtemadlin is an MDM2 inhibitor compound of the disclosure. Navtemadlin was administered intratumorally as 20 μL of 100 mg/mL navtemadlin Q3D×9, 20 μL of 100 mg/mL navtemadlin Q7D×5, 20 μL of 100 mg/mL navtemadlin Q14D×3, and 20 μL of 25 mg/mL navtemadlin QD×31.

The data show that administration intratumorally of 20 μL of 100 mg/mL navtemadlin Q3D×9, 20 μL of 100 mg/mL navtemadlin Q7D×5, 20 μL of 100 mg/mL navtemadlin Q14D×3, and 20 μL of 25 mg/mL navtemadlin QD×31 all resulted in significant tumor volume reduction while not significantly affecting % body weight change.

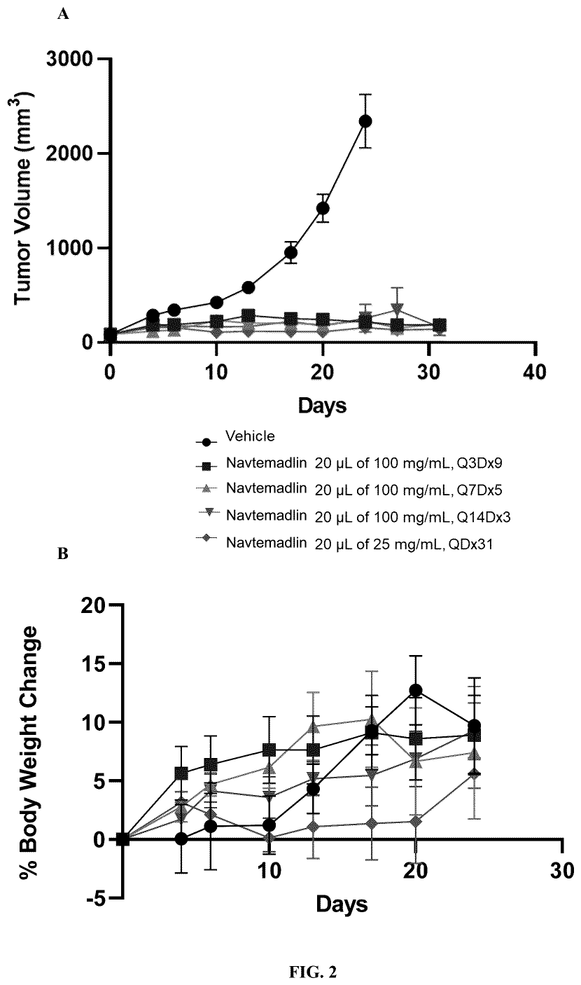

FIG. 2 panels A and B show the effects of intratumoral dosing regimens of navtemadlin in SJSA1 tumors, such as on tumor volume (mm 3) and % body weight change. TABLE 4 shows % tumor inhibition when navtemadlin was delivered intratumorally compared to the vehicle.

TABLE 4

| Navtemadlin/Regimen | % TGI Day 24 |
|---|---|
| Vehicle | — |
| Q3Dx21 IT | 94% |
| Q7Dx21 IT | 94% |
| Q14Dx21 IT | 94% |
| QDx21 PO | 94% |

### Example 3: Dosing Regimen and Formulations for Intratumoral Administration

The effect of intratumoral administration of navtemadlin is tested in a once monthly dose, 2 month dose, and a 2-week dose in NUGC3 mice. Tumor volume reduction (mm³) and % body weight change are measured.

A formulation of navtemadlin is prepared for intratumoral administration. The formulation comprises poly(lactic-co-glycolic acid) polymer, which is an extended release formulation with a FDA-approved dose of up to 500 mg/dose.

### Example 4: Formulation for Intratumoral Administration

Poly(lactic-co-glycolic acid), (250 mg, PLGA, Ashland, Viatel DLG 5002A part 952375 lot 0002515384) and MDM2 inhibitor navtemadlin (100 mg) were added to a 4 mL vial. N-Methyl-2-pyrrolidone (650 mg, NMP, Sigma Aldrich part 328634-1L lot SHBC9583) was added and the mixture was vortexed for approximately 1-5 min until mixture could be stirred on a magnetic stir-plate. The mixture was stirred for 30-120 min until polymer and compound was fully dissolved. After the mixture was fully dissolved, sterile filtered into a sterile septum vial using a 0.22micron nylon filter. Concentration of 100 mg/mL (85-115% accuracy) was confirmed by HPLC analysis. This solution, when injected into the tumor, solidified into a semi-solid and formed an in-situ gel implant.

1. A method of treating a tumor in a subject in need thereof comprising administering to the subject a therapeutically-effective amount of a MDM2 inhibitor, wherein the administering is by intratumoral injection at most once every 3 days.

2. The method of claim 1, wherein the tumor is breast cancer.

3. The method of claim 1, wherein the tumor is ovarian cancer.

4. The method of claim 1, wherein the tumor is pancreatic cancer.

5. The method of claim 1, wherein the tumor is lung cancer.

6. The method of claim 1, wherein the administering by intratumoral injection occurs once every 3 days.

7. The method of claim 1, wherein the administering is by intratumoral injection occurs once every 7 days.

8. The method of claim 1, wherein the administering is by intratumoral injection occurs once every 14 days.

9. The method of claim 1, wherein the administering by intratumoral injection occurs once a month.

10. The method of claim 1, wherein the administering by intratumoral injection occurs once every two months.

11. The method of claim 1, wherein the administering reduces a tumor volume by at least about 20%.

12. The method of claim 1, wherein the administering reduces a tumor volume by at least about 50%.

13. The method of claim 1, wherein the therapeutically-effective amount is from about 0.05 mg to about 50 mg.

14. The method of claim 1, wherein the therapeutically-effective amount is about 5 mg.

15. The method of claim 1, wherein the therapeutically-effective amount is about 25 mg.

16. The method of claim 1, wherein the subject is human.

17. The method of claim 1, wherein the MDM2 inhibitor is a small molecule.

18-19. (canceled)

20. The method of claim 1, wherein the MDM2 inhibitor is selected from: 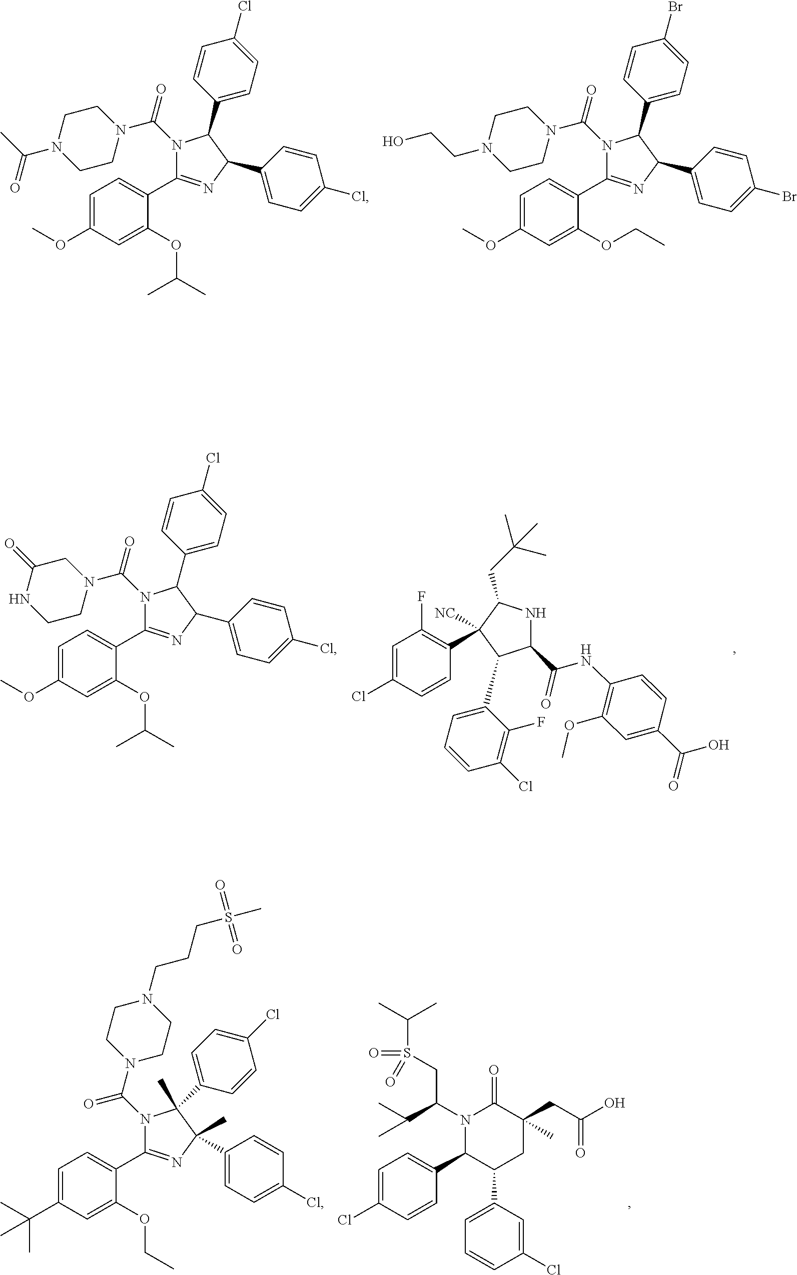 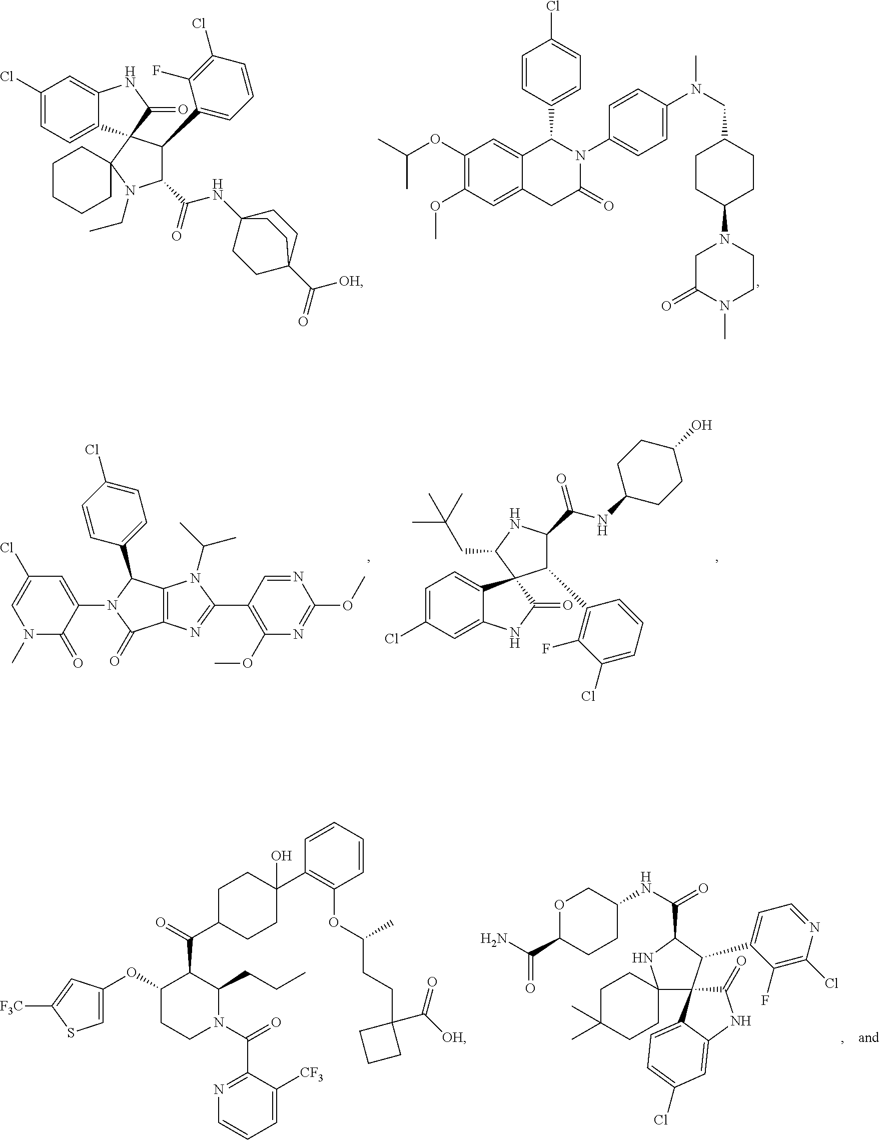 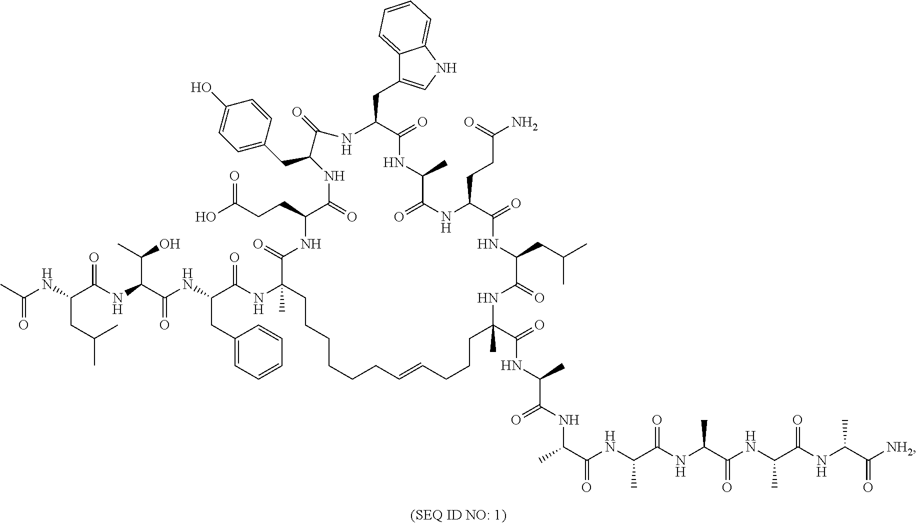 [SMILES: CC(=O)N[C@@H](CC(C)C)C(=O)N[C@H](C(=O)N[C@@H](Cc1ccccc1)C(=O)N[C@]1(C)CCCCCC/C=C/CCC[C@@](C)(C(=O)N[C@@H](C)C(=O)N[C@@H](C)C(=O)N[C@@H](C)C(=O)N[C@@H](C)C(=O)N[C@@H](C)C(=O)N[C@H](C)C(N)=O)NC(=O)[C@H](CC(C)C)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]
or a pharmaceutically-acceptable salt thereof.

21-117. (canceled)


SEQUENCE LISTING (1 sequence)

SEQ ID NO: 1 - protein, 17 aa, synthetic construct
  SITE 1: note = N-terminal acetylation
  MOD_RES 4: note = alpha-((E)-undec-4-en-1-yl)-L-alanine
  CROSSLNK 4..11: note = terminal carbon of (E)-undec-4-ene side chain of residue 4 bound to alpha position of residue 11
  MOD_RES 17: note = D-amino acid
  SITE 17: note = C-terminal NH2
LTFAEYWAQLAAAAAAA